SEQUENCE LISTING (6 sequences)

SEQ ID NO: 1 - DNA, 18 bp, Artificial Sequence
  Description of Artificial Sequence Synthetic primer
tcctacaccacaccaaac

SEQ ID NO: 2 - DNA, 19 bp, Artificial Sequence
  Description of Artificial Sequence Synthetic primer
ctccaatctctgcctatcc

SEQ ID NO: 3 - DNA, 18 bp, Artificial Sequence
  Description of Artificial Sequence Synthetic primer
cctctgcgatgctcttcc

SEQ ID NO: 4 - DNA, 23 bp, Artificial Sequence
  Description of Artificial Sequence Synthetic primer
tcacacttatactggtcaaatcc

SEQ ID NO: 5 - DNA, 18 bp, Artificial Sequence
  Description of Artificial Sequence Synthetic primer
tcaacggcacagtcaagg

SEQ ID NO: 6 - DNA, 19 bp, Artificial Sequence
  Description of Artificial Sequence Synthetic primer
actccacgacatactcagc

(19) United States
(12) Patent Application Publication
(10) Pub. No.: US 2011/0206720 A1
(43) Pub. Date: Aug. 25, 2011

(54) ANTI-INFLAMMATORY USAGE OF ANTRODIA CINNAMOMEA FRUIT BODY DERIVATIVES

(75) Inventor: Sheng-Yang WANG, Taipei Country (TW)

(73) Assignee: Taiwan Leader Biotech Corp, Taipei Country (TW)

(21) Appl. No.: 12/711,209

(22) Filed: Feb. 23, 2010

(51) Int. Cl. A61K 36/06; C07C 43/215; A61P 29/00; C12N 5/00

(52) U.S. Cl. 424/195.15; 568/658; 435/375

(57) ABSTRACT

The present invention is related to the usage of Antrodia cinnamomea fruit body derivatives. The fruit body derivatives comprise acetic acid extract, acetic ether extract and Antrocamphin A. The acetic acid extract, acetic ether extract and Antrocamphin A can reduce the expression of iNOS and COX-2 and therefore inhibit the production of pro-inflammatory molecules.

### BACKGROUND OF INVENTION

1. Field of the Invention

The present invention relates generally to Antrodia cinnamomea fruit body derivatives, and more particularly to the anti-inflammatory usage of Antrodia cinnamomea fruit body derivatives.

2. Description of Related Art

Inflammation is a key feature under many pathological states. Taking bacterial infection as an example, the lipopolysaccharide (LPS, also known as endotoxin) on the surface of bacteria will activate macrophages in the hosts. Under normal situations, this activation is conducive to the activation of the immune system of the host and thus killing pathogenic bacteria. In some abnormal physiological responses (possibly accompanying diseases or the major cause of a disease), the activated macrophages might elicit the expression of too many NO and cause host cells to die. Therefore, under many pathological states, inhibition of inflammatory responses is an important part for medical treatment.

For some inflammatory stimuli, the production of Tumor Necrosis Factor-α (TNF-α), NO and prostaglandin-E₂ (PGE₂, produced by arachidonic acid through COX pathway metabolism is an important part of the immune response. For example, said vectors have been detected excessively in the inductions of septic and hemorrhagic shock, rheumatoid arthritis and arthrosclerosis. Thus, the new kind of drug is designed to inhibit the genetic expression of iNOS and COX-2, deactivate main receptors or ferments of LPS inducing message transmission, and thus suppressing the production of NO and prostaglandin-E₂.

Antrodia cinnamome is a kind of fungus which is deemed as a very precious traditional Chinese medicine. It has already been proven in many scientific studies that methanol extract and hot water extract of Antrodia cinnamome mycelium, and acetic acid extract, acetic ether extract and Antrocamphin A of fruit body derivatives has good antitumor activity. And some studies also pointed out, the mycelium and fruit body derivatives of Antrodia cinnamome contain triterpenoid, which might be the compounds with the potential anti-inflammatory ability. However, further studies still need for knowing the mechanism and physiological activity of Antrodia cinnamome and triterpenoid it contains.

### SUMMARY OF THE INVENTION

The primary objective of the present invention is to provide Antrodia cinnamomea fruit body derivatives and its usage for anti-inflammation.

In order to achieve this objective, the present invention provides a kind of extract from Antrodia cinnamomea fruit body derivatives, which is obtained in the following steps: provide dry Antrodia cinnamomea fruit body; extract with ethanol at a certain temperature.

Another objective of the present invention is to provide extract from Antrodia cinnamomea fruit body by the following steps: provide dry Antrodia cinnamomea fruit body; extract with ethanol at a certain temperature to get the acetic acid extract; concentrate said acetic acid extract to get a concentrated product; partition the concentrated product by acetic ether and water to produce the acetic ether extract.

Preferably, said drying is in the way of freeze drying.

Preferably, said temperature is 42˜45° C.

Preferably, said ethanol is of 95% volume percentage concentration.

Preferably, said concentration is vacuum concentration.

Preferably, said acetic ether is of 50% volume percentage concentration.

The present invention further provides a usage of said extract for inhibiting the production of pro-inflammatory molecules.

Preferably, said pro-inflammatory molecules are NO or prostaglandin-E₂ (PGE₂).

Preferably, said extract reduces the expression of iNOS and COX-2 and therefore inhibits the production of said NO and pro-inflammatory molecules.

The present invention provides antrocamphin A for inhibiting the production of NO.

Preferably, said antrocamphin A reduces the expression of iNOS and therefore inhibits the production of said NO.

The present invention provides another antrocamphin A for inhibiting the production of prostaglandin-E₂ (PGE₂).

Preferably, said antrocamphin A reduces the expression of COX-2 and therefore inhibits the production of said prostaglandin-E₂ (PGE₂).

The present invention provides a method for inhibiting the production of pro-inflammatory molecules, which make the tested object in contact with any extract or antrocamphin A in Item 17 of the patent application.

Preferably, said pro-inflammatory molecules are NO or prostaglandin-E₂ (PGE₂).

As stated above, the present invention relates generally to the anti-inflammatory usage of Antrodia cinnamomea fruit body derivatives, and more particularly to revealing the physiological activity and action mechanism of Antrodia cinnamomea fruit body derivatives for anti-inflammation.

### BRIEF DESCRIPTION OF THE DRAWINGS

FIG. 1 shows the inhibition rates of NO by acetic acid extract, acetic ether extract and water-soluble fraction of the present invention.

FIG. 2A shows the expression of iNOS inhibited by acetic acid extract of the present invention.

FIG. 2B shows the expression of COX-2 inhibited by acetic acid extract of the present invention.

FIG. 3 shows the inhibition rate of NO by 17 fractions of acetic ether extract of the present invention.

FIG. 4A shows the expression of mRNA of iNOS inhibited by antrocamphin A.

FIG. 4B shows the expression of mRNA of COX-2 inhibited by antrocamphin A.

FIG. 5A shows the expression of iNOS inhibited by antrocamphin A.

FIG. 5B shows the expression of COX-w inhibited by antrocamphin A.

### DETAILED DESCRIPTION OF THE INVENTION

The present invention is related to the usage of Antrodia cinnamomea fruit body derivatives for inhibiting the production of pro-inflammatory molecules, and thus revealing extraordinary anti-inflammation ability. Said “derivatives” indicate the extracts and compounds of the chemical compositions of Antrodia cinnamomea fruit body; more specifically, the “derivatives” of the present invention are acetic acid extract, acetic ether extract and Antrocamphin A.

Lipopolysaccharide (LPS) are large molecules comprising of a lipid and a polysaccharide joined by a covalent bond; they are the major component of the outer membrane of Gram-negative bacteria, and act as endotoxins and elicit strong inflammatory responses in the body of hosts, which are generally immune responses activated by macrophages. Therefore, the present invention takes LPS eliciting inflammatory responses in the bodies of experimental mice or macrophage as the model for discussion about the role of Antrodia cinnamomea fruit body derivatives in the anti-inflammatory responses.

The experiments in the following are taken as examples to know the details and connotations of the present invention, but not included in the scope of the patent applied for the present invention.

### Experiment I

### Experimental Set-up

### [Materials]

The Antrodia cinnamomea fruit body is the wild one growing in Liugui, Kaohsiung, Taiwan. The FBS (fetal bovine serum) was purchased from Gibco BRL (Invitrogen, Grand Island, N.Y.). DMSO, penicillin, (lipopolysaccharide, Escherichia coli 0127:138/LPS), MTT (3-(4,5-dimethylthiazol-2-yl)-2,5-diphenyltetrazolium bromide) and Griess reagent were bought from Sigma-Aldrich (St Louis, Mo.). All chemical medicines and solvents used by the invention are of reagent or HPLC grade.

### [Experimental Mice]

The experimental mice are four-week-old male ICR mice (about 25-28 g) from BioLasco (Taipei, Taiwan). Before the experiment, the mice are fed for at least a week at the temperature 25±2° C. and the relative humidity 55±5%, with 12 hours for light/dark period each (more specifically, 6:00 in the morning to 18:00 in the afternoon are the light period), and with abundant supply of feedstuff and water so that these mice can adapt to the environment. The experimental use of animals for the experiment follows the Guide for the Care and Use of Laboratory Animals and relevant animal protection laws of Taiwan, and is approved by local moral committee.

### [Cell Culture]

The invention chooses the macrophage RAW 267.4 of mice for the discussion about the production of NO and prostaglandin-E₂ (PGE₂). For the culture of RAW 267.4 cell, firstly inoculate RAW 267.4 macrophages growing in the culture plate (75 cm²) in 96-well plate in the density of 2×10⁵ cells/well and then culture them in the incubator (37° C., 5% CO₂). DMEM culture medium is selected according to the recommendation of ATCC (American TypeCulture Collection), with 10% fetal bovine serum (FBS), penicillin (100 units/ml) and streptomycin (100 g/ml) added.

### [Western Blot Analysis]

After getting the total protein of macrophages, add the equal amount of protein in 5˜7% SDS-PAGE, resolve for 90 m at 300 mA, blotting (100V, 1 hour) proteins resolved in different sizes to polyvinylidene difluoride (PVDF, Immobilon, Millipore, Bedford, Mass.), and then put PVDF in the blocking buffer (10% w/v skim milk powder in TBST) for 1 hour.

Then, dip PVDF in the anti-iNOS solution (1:1000, Cayman Chemicals) and clean twice with 0.1% TBST buffer (TBS buffer containing 0.1% Tween 20) to clear the non-specific bonded antibody and antigen.

Next, dip PVDF in the anti-rabbit secondary antibodies solution with horseradish peroxidase, detect the fluorescence density of each group by enhanced chemiluminescene regents (ECL, Pierce), and take the expression of β-actin as the reference group.

### [RT-PCR (Reverse Transcription-Polymerase Chain Reaction)]

Trizol reagent (Invitrogen Life Technologies, Carlsbad, Calif., USA) is used to extract the total RNA of macrophages. mRNA of iNOS, COX-2 and G3PDH is reversely transcribed to be cDNA by the RT-PCR, and then the expression of iNOS, COX-2 and G3PDH are quantified with the real time-PCR (Applied Biosystems).

Said real time-PCR detects the PCR products by DNA binding SYBR Green. The thermal cycles are: 95° C., 5 min, 1 min for 40 cycles at 95° C., 45 s for 55° C., and 30 s for 72° C. The sequence of primers is: iNOS forward 5′-TCC TAC ACC ACA CCA AAC-3′; iNOS reverse 5′-CTC CAA TCT CTG CCT ATC C-3′; COX-2 forward 5′-CCT CTG CGA TGC TCT TCC-3′, COX-2 reverse 5′-TCA CAC TTA TAC TGG TCA AAT CC-3′, G3PDH forward 5′-TCA ACG GCA CAG TCA AGG-3′; G3PDH reverse 5′-ACT CCA CGA CAT ACT CAG C-3′. G3PDH are housekeeping genes which are genes with stable and large number of expression in cells and are used to standardize the expression of iNOS and COX-2 in the experiment.

### Experiment II

### The Preparation of Acetic Acid Extract and Acetic Ether Extract from Antrodia cinnamomea Fruit Body

Firstly, the Antrodia cinnamomea fruit body is dried in the way of freeze drying and then extracted 580 g at 42˜45° C. with 95% ethanol, obtaining the acetic acid extract (ACE). Said drying can be in any way in familiar areas, without any restrictions.

Then, said acetic acid extract is concentrated under vacuum by rotary evaporator to get 183.9 g concentrated product, which is partitioned to be acetic ether layer and water layer. The water layer is the ACE-water, and the acetic ether layer is the ACE-EA (acetic ether extract) of the present invention.

### Experiment III

### Testing ACE, ACE-EA and ACE-water Extracted in Experiment II for their Inhibitions of NO Activity: Macrophage Raw 267.4

In the experiment, macrophage RAW 267.4 is employed to test ACE, ACE-EA and ACE-water extracted in Experiment II for their inhibitions of NO activity.

Firstly, RAW 267.4 is cultured as detailed in Experiment I and divided into three groups: group 1 (ACE), group 2 (ACE-EA) and group 3 (ACE-water). For each group, add said ACE, ACE-EA and ACE-water of different concentrations (1, 10, 25, 50 g/ml) in the culture medium, add LPS to elicit RAW267.4 producing NO, and measure the content of nitrite after 24 hours by Griess reaction to indirectly measure the amount of NO. The experimental design is as shown in Table 1 as follows:

TABLE 1

| Groups | ACE | ACE-EA | ACE-water |
|---|---|---|---|
| LPS (1 g/ml) | + | + | + |
| ACE | + | − | − |
| (1, 10, 25, | | | |
| 50 g/ml) | | | |
| ACE-EA | − | + | − |
| (1, 10, 25, | | | |
| 50 mg/ml) | | | |
| ACE-water | − | − | + |
| (1, 10, 25, | | | |
| 50 mg/ml) | | | |

“+” means being added;
“−” means not being added.

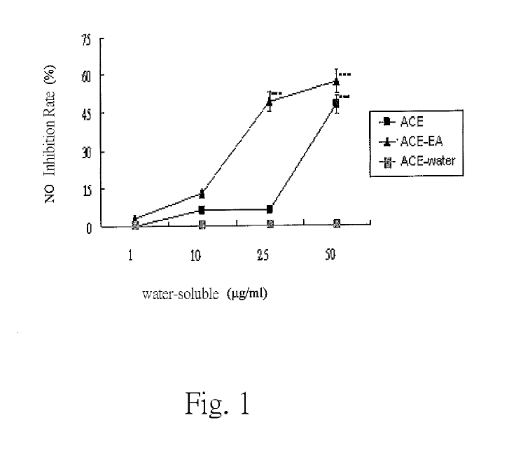

Please see FIG. 1, which shows the amount of NO measured by Griess reaction and the results of calculated inhibition rates of NO. From the figure it is known that ACE and ACE-EA obtained in Experiment II exhibit the inhibition of NO activity, especially well-performed at 50 g/ml.

### Experiment IV

### Testing ACE's Inhibition of NO Activity: Experiment with Live Mice

The experiment tests the ACE's inhibition of NO activity with mice prepared in Experiment I as the laboratory animals.

### [Experiment Design]

Firstly, 6 mice are put into a group and there are totally 6 groups as shown in Table 2. Before LPS (5 g/kg) is injected, each group of mice are injected with ACE (100, 300, 500 mg/kg) obtained in Experiment II or curcumin by intraperitoneal injection or not injected. Here, the control group is injected with DMSO only. Curcumin is a known anti-inflammation drug and is used as the positive reference group in this experiment. After being injected with LPS, mice are etherized and decapitated 12 hours later.

TABLE 2

| Groups | LPS (5 g/ml) | ACE | curcumin |
|---|---|---|---|
| ACE 100 | + | + (100 mg/kg) | − |
| ACE 300 | + | + (300 mg/kg) | − |
| ACE 500 | + | + (500 mg/kg) | − |
| curcumin | + | − | + (100 mg/kg) |
| negative | + | − | − |
| reference group | | | |
| control group | − | − | − |

“+” means being added;
“−” means not being added.
Not LPS but DMSO is used for the control group.

### [Results]

After blood sampling by orbital bleeding or heart puncture, bloods are put in the test tubes with EDTA (ethylenediamine tetra-acetic acid), centrifuged 500 g for 10 min at 4° C., and serums are collected for measuring the concentration of NO in serums by Griess reaction. The results are as shown in Table 3:

TABLE 3

| Groups | Drug/Dose | NO (M) |
|---|---|---|
| negative | LPS (5 g/ml) | 37.79 ± 1.87 |
| reference | | |
| group | | |
| ACE 100 | LPS (5 g/ml) | 22.6 ± 0.44 |
| | ACE (100 mg/kg) | |
| ACE 300 | LPS (5 g/ml) | 15.04 ± 0.3 |
| | ACE (300 mg/kg) | |
| ACE 500 | LPS (5 g/ml) | 8.79 ± 0.11 |
| | ACE (500 mg/kg) | |
| curcumin | LPS (5 g/ml) | 10.79 ± 0.27 |
| | curcumin (100 mg/kg) | |

As shown in Table 3, the ACE obtained in Experiment II significantly inhibits the production of NO and has the similar effect as curcumin does.

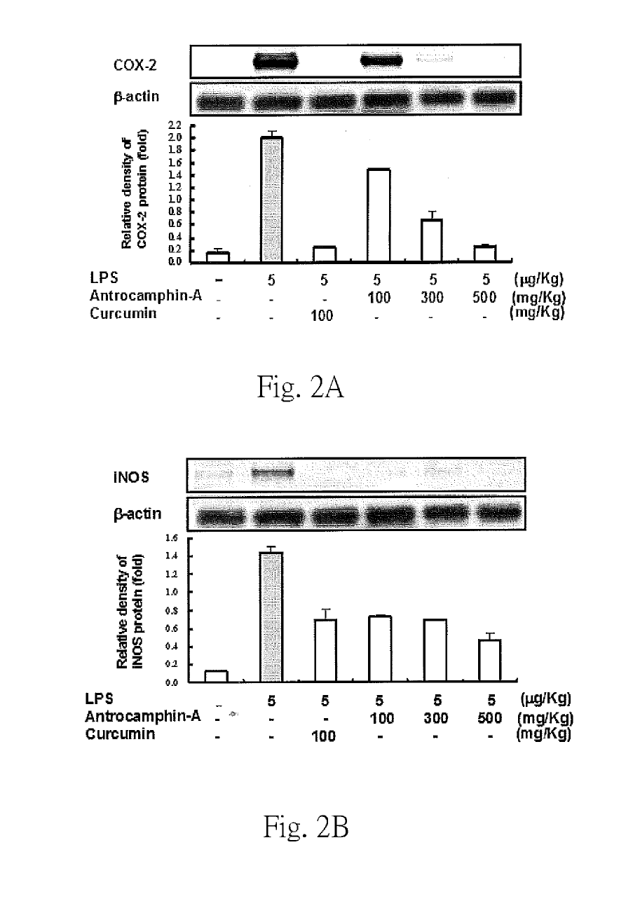

In addition, the livers of sacrificed experimental mice are taken out to extract the total protein by known experimental approach. The concentration of proteins is quantified with Bradford method at the absorbance at 595 nm. The expression of iNOS and COX-2 in total protein are measured by the western blot analysis (see the paragraph of Western blot analysis in Experiment I). From the results in FIG. 2A and FIG. 2B, it is known that the ACE obtained in Experiment II significantly reduces the expression of iNOS and COX-2. This can explain why ACE obtained in Experiment II can inhibit the production of NO.

### Experiment V

### Separation of Antrocamphin A

The ACE-EA extracted in Experiment II is divided into 17 fractions by chromatography according to concentration gradients of mobile phase. The stationary phase utilized by said chromatography is silica gel (60-80 mesh) and the mobile phase is n-Hexane/acetic ether. Said concentration gradients are n-Hexane: acetic ether=95:5, 90:10, 85:15, 80:20, 70:30, 60:40, 50:50, 40:60, 0:100 as the mobile phase for carrying out the chromatography.

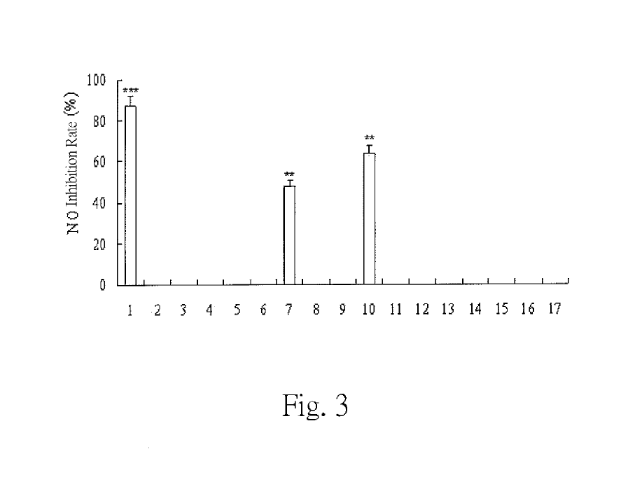

NO activity inhibition tests are conducted on RAW 267.4 macrophages (see reference in Cell culture, Experiment I) to 17 fractions respectively. Firstly, divide RAW 267.4 cells into 17 groups, add 17 fractions (50 g/ml) in the culture medium, and then add LPS (1 g/ml) to elicit the production of NO. 24 hours later, Griess reaction is conducted to measure the content of nitrites and thus indirectly measuring the production of NO and calculating the inhibition rate of NO. Please see FIG. 3 for the experimental results, which indicate that the 1st, 7th and 10th fractions have the inhibition rates of 87%, 48% and 64%.

Said 1st fraction is further separated and purified by HPLC to get its ingredients (stationery phase: silica gel column; mobile phase: n-Hexane/acetic ether solution with the volume ratio of 85:15; flow speed: 3 ml/min; UV wavelength: 254 nm). The molecular weight of ingredient of the highest content is 247.14 in mass spectrometry analysis and its molecular formula is C₁₅H₁₉O₃, whose structure is further confirmed by 1H-NMR as follows:

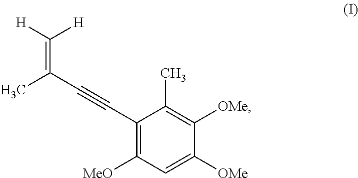

It is proved to be the compound: Antrocamphin A.

### Experiment VI

### Test of Antrocamphin A Inhibiting the Production of Pro-Inflammatory Molecules: Macrophages RAW 267.4

In this experiment, macrophages RAW 267.4 are employed to test the inhibition of pro-inflammatory molecules by Antrocamphin A obtained in Experiment V.

In the experiment of production of NO, firstly RAW 267.4 cells are cultured as Experiment I states, handled for 1 hour in antrocamphin A (1, 5, 10, 20 g/ml) or curcumin (10 g/ml), and then processed for 24 hours with LPS (1 g/ml). Then, Griess reaction is conducted to measure the content of nitrites and thus indirectly obtaining the production of NO.

In the experiment for measuring the production of prostaglandin-E₂ (PGE₂), firstly RAW 267.4 cells are cultured as Experiment I states and 500 ml aspirin is added in the culture medium for 3 hours so that the endogenous COX-1 is deactivated. Then, they are handled for 1 hour in antrocamphin A (1, 5, 10, 20 g/ml) or curcumin (10 g/ml) and processed for 16 hours with LPS (1 g/ml). Finally, supernatant liquid in the cell culture is measured by ELISA kit (Cayman Chemicals) so as to get the production of prostaglandin-E₂ (PGE₂) in endogenous arachidonic acid.

The group setting of this experiment is as shown in Table 4:

TABLE 4

| Groups | LPS (1 g/ml) | Antrocamphin A | Curcumin |
|---|---|---|---|
| Antrocamphin | + | + (1 g/ml) | − |
| A-1 | | | |
| Antrocamphin | + | + (5 g/ml) | − |
| A-5 | | | |
| Antrocamphin | + | + (10 g/ml) | − |
| A-10 | | | |
| Antrocamphin | + | + (20 g/ml) | − |
| A-20 | | | |
| Curcumin-10 | + | − | + (10 g/kg) |
| negative | + | − | − |
| reference group | | | |
| control group | − | − | − |

“+” means being added;
“−” means not being added.
Not LPS but DMSO is given to the control group.

In addition, the familiar MTT is utilized to test the toxicity of Antrocamphin A to cells. Said MTT is a method for detecting cell survival rate or cell proliferation in biology on principle that succinate dehydrogenase in the mitochondria of live cell metabolizes tetrazolium of MTT to be blue products. After DMSO is added to resolve the blue metabolized products accumulated in cells, the amount of blue metabolized products is measured by spectrometer to indirectly calculate the number of live cells.

The experimental results are as shown in Table 5:

TABLE 5

| | | Production of | |
|---|---|---|---|
| | Production of | prostaglandin-E₂ | Cell survival |
| Groups | NO (M) | (PGE₂) | rate (%) |
| negative | 17.43 ± 0.42 | 429.04 ± 28.47 | 100 ± 6.45 |
| reference group | | | |
| antrocamphin | 18 ± 0.36 | 411.5 ± 20.92 | 93 ± 4.13 |
| A-1 | | | |
| antrocamphin | 14.68 ± 0.54 | 360.9 ± 23.95 | 92 ± 5.49 |
| A-5 | | | |
| antrocamphin | 11.14 ± 0.18 | 237.96 ± 83.85 | 89 ± 3.22 |
| A-10 | | | |
| antrocamphin | 7.27 ± 0.18 | 24.62 ± 15.36 | 79 ± 3.54 |
| A-20 | | | |
| Curcumin-10 | 16.69 ± 0.2 | 6.82 ± 5.2 | 76 ± 4.6 |

From the results we know that antrocamphin A can reduce the production of pro-inflammatory molecules and the concentration for significant effect is 20 g/ml, at which antrocamphin A can inhibit cytotoxicity.

In addition, total RNA and total protein are extracted from each group of macrophages. According to the paragraphs of RT-PCR and western blot analysis in Experiment I, the expression of iNOS and COX-2 are evaluated from the perspectives of mRNA and protein.

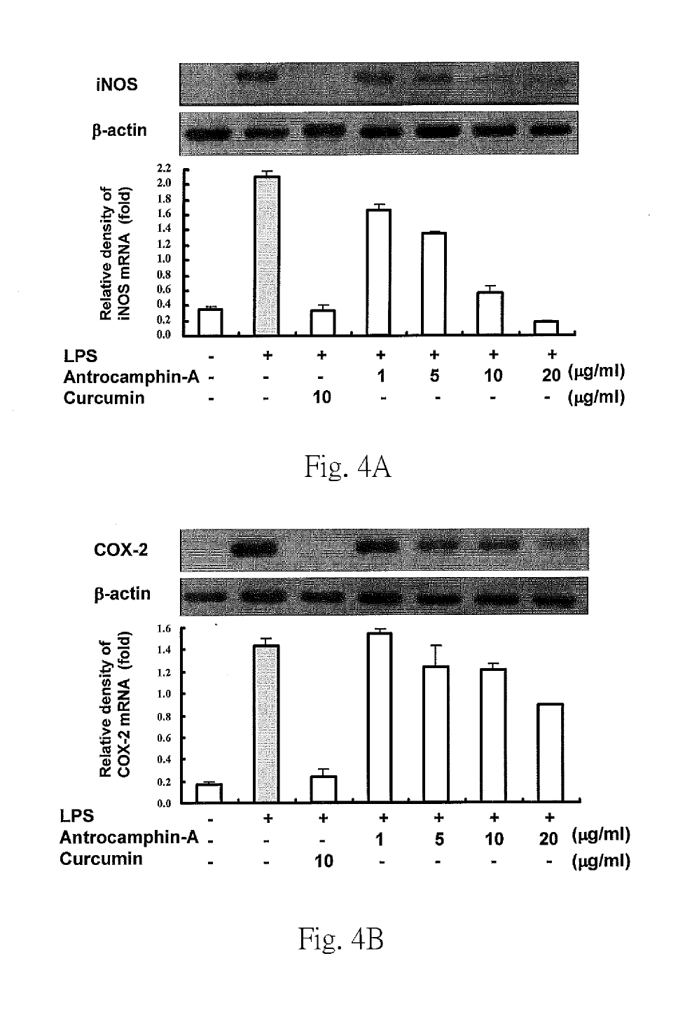

FIGS. 4A and 4B depict the expression of mRNA of iNOS and COX-2 respectively. Accordingly, the expression of mRNA of iNOS and COX-2 have dose-dependent relation with antrocamphin A. The higher dose of antrocamphin A can effectively reduce the amount of mRNA of iNOS and COX-2.

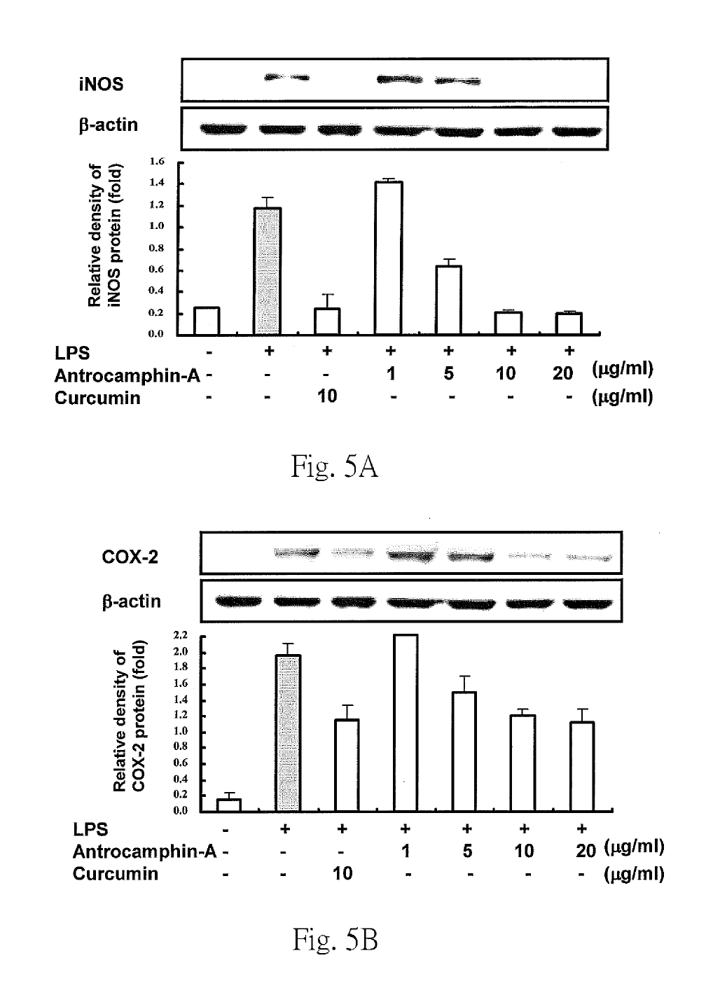

FIGS. 5A and 5B depict the expression of iNOS and COX-2 respectively. From the figures it is known that the expression of iNOS and COX-2 have dose-dependent relation with antrocamphin A. The higher dose of antrocamphin A can effectively reduce the expression of iNOS and COX-2, especially at 20 g/ml.

To sum up, from the perspective of either genetic transcription or protein expression, antrocamphin A can reduce the expression of iNOS and COX02, and therefore inhibit the production of pro-inflammatory molecules such as NO and prostaglandin-E₂.

1. An extract from Antrodia cinnamomea fruit body derivatives, which is obtained in the following steps:
provide dry Antrodia cinnamomea fruit body;
extract with ethanol at a certain temperature.

2. The extract from Antrodia cinnamomea fruit body derivatives as claimed in claim 1, wherein said drying is freeze drying.

3. The extract defined in claim 1, wherein said temperature is 42˜45° C.

4. The extract from Antrodia cinnamomea fruit body derivatives as claimed in claim 1, wherein said ethanol is of 95% volume percentage concentration.

5. An extract from Antrodia cinnamomea fruit body derivatives obtained in these steps:
provide dry Antrodia cinnamomea fruit body;
extract with ethanol at a certain temperature to get the acetic acid extract;
concentrate said acetic acid extract to get a concentrated product;
partition the concentrated product by acetic ether and water to produce said acetic ether extract.

6. The extract from Antrodia cinnamomea fruit body derivatives as claimed in claim 5, wherein said drying is freeze drying.

7. The extract from Antrodia cinnamomea fruit body derivatives as claimed in claim 5, wherein said concentration is vacuum concentration.

8. The extract from Antrodia cinnamomea fruit body derivatives as claimed in claim 5, wherein said temperature is 42˜45° C.

9. The extract from Antrodia cinnamomea fruit body derivatives as claimed in claim 5, wherein said ethanol is of 95% volume percentage concentration.

10. The extract from Antrodia cinnamomea fruit body derivatives as claimed in claim 5, wherein said acetic ether is of 50% volume percentage concentration.

11. The usage of the extract from Antrodia cinnamomea as claimed in claim 1 for inhibiting the production of pro-inflammatory molecules.

12. The usage of the extract from Antrodia cinnamomea as claimed in claim 11, wherein said pro-inflammatory molecules are NO or prostaglandin-E₂ (PGE₂).

13. The usage of the extract from Antrodia cinnamomea as claimed in claim 12, wherein said extracts reduce the expression of iNOS and COX-2 and therefore inhibit the production of said NO or prostaglandin-E₂ (PGE₂).

14. An antrocamphin A for inhibiting the production of NO.

15. The usage of antrocamphin A as claimed in claim 14, wherein said antrocamphin A reduces the expression of iNOS to inhibit the production of said NO.

16. An antrocamphin A for inhibiting the production of prostaglandin-E₂ (PGE₂).

17. The usage of antrocamphin A as claimed in claim 16, wherein said antrocamphin A reduces the expression of COX-2 to inhibit the production of said prostaglandin-E₂ (PGE₂).

18. A method for inhibiting the production of pro-inflammatory molecules, which make the tested object in contact with any extract or antrocamphin A in claim 17.

19. The method of inhibiting the production of pro-inflammatory molecules as claimed in claim 18, wherein said pro-inflammatory molecules are NO or prostaglandin-E2 (PGE2).

20. The usage of the extract from Antrodia cinnamomea as claimed in claim 5 for inhibiting the production of pro-inflammatory molecules.
